(19) United States
(12) Patent Application Publication
(10) Pub. No.: US 2004/0151702 A1
(43) Pub. Date: Aug. 5, 2004

(54) Production and use of a suspension composition comprising an ultrasound contrast medium

(76) Inventor: Rainer Marksteiner, Schwaz (AT)

Correspondence Address: DUANE MORRIS, LLP, IP DEPARTMENT, ONE LIBERTY PLACE, PHILADELPHIA, PA, 19103-7396, US

(21) Appl. No.: 10/475,631

(22) PCT Filed: Apr. 19, 2002
(86) PCT No.: PCT/EP02/04363

(30) Foreign Application Priority Data
Apr. 20, 2001 (DE) 10119522.2

(51) Int. Cl. A61K 48/00; A61K 39/00

(52) U.S. Cl. 424/93.7; 424/277.1; 424/93.21

(57) ABSTRACT

A suspension composition is described, which contains at least one functional component effective in biological tissue and which forms a dosage form for administration into the tissue, whereby the functional component is suspended or dissolved in a suspension fluid, the functional component contains at least one active ingredient, biological cells, bacteria, viruses and/or biological or biocompatible fillers, and an ultrasound contrast medium is contained in the suspension fluid. Uses of the suspension composition, in particular for injection into biological tissue, are also disclosed.

The present invention pertains to a material composition, which contains one or more ultrasound contrast media, to processes for the production and the use of the material composition, devices for implementing the processes and uses of ultrasound contrast media and of suspensions of biological materials, such as, e.g., of cell suspensions or suspensions of natural proteins.

A large number of processes for cell culture engineering which concern the culturing of certain cells or the colonization of polymers with cells, which are provided for later administration into the body, have been known for decades from the literature. Cell culture techniques, administration forms of cells and medications, their use for the treatment of diseases and techniques for the culturing and the therapeutic use of differentiated cells are described (see, e.g., U.S. Pat. No. 4,645,669). The encapsulation of live tissue and cells with a biocompatible membrane before administration has become known from U.S. Pat. No. 4,673,566 and U.S. Pat. No. 4,689,293.

U.S. Pat. No. 5,336,263 discloses the treatment of gastroesophageal reflux with an injection of microparticles. U.S. Pat. No. 5,662,609 discloses a method and a device for the treatment of local diseases in hollow organs and other tissues. A device, not described in detail, is defined, which has an elongated tubular shaft and a distal region for introduction into the patient and a proximal region that is not introduced into the body. By means of this device, monomers, prepolymers, polymers and a therapeutic agent are administered into the body. The treatment of ureterovesical reflux, incontinence as well as diseases of the digestive tract, the genitourinary tract and the chest cavity with injections of muscle cells of the urinary bladder and cell-polymer suspensions is described in U.S. Pat. No. 5,667,778 and U.S. Pat. No. 5,976,526. Methods, with which cell-polymer suspensions are administered into the body, so that new tissue is formed, are revealed in U.S. Pat. No. 5,709,854. The technique of producing breast tissue has become known from U.S. Pat. No. 5,716,404. Injectable hydrogel compositions that contain cells and are administered into the body are described in U.S. Pat. No. 6,129,761.

Imaging ultrasound techniques have been widely used in diagnostics in the last 20 years. In this case, ultrasound contrast media increasingly represent an integrative component of the diagnostic instrumentation. Thus, vessels, the supply of blood to vessels, but also tumors or structures, which otherwise can only be visualized with great difficulty, can be better visualized in ultrasound. The ultrasound contrast media used up to now were almost exclusively injected into the blood stream to make possible an improved examination of the supply of blood to vessels, organs or tumors. These substances are then excreted above all in the liver or the kidneys.

A large number of different substances (ultrasound contrast media), which have ultrasound contrast-enhancing action, has become known from scientific publications and the patent literature. Already in the 1970s and the beginning of the 1980s, methods were patented, with which ultrasound contrast could be enhanced (see, e.g., U.S. Pat. No. 3,640,271). In further succession, numerous different ultrasound contrast media, production methods and sonographic methods were developed for achieving a better ultrasound contrast and a better image quality (U.S. Pat. No. 4,466,442, U.S. Pat. No. 4,657,756, U.S. Pat. No. 5,141,738, U.S. Pat. No. 5,147,631, U.S. Pat. No. 5,333,613, U.S. Pat. No. 5,352,435, U.S. Pat. No. 5,501,863, U.S. Pat. No. 5,540,909, U.S. Pat. No. 5,558,854, U.S. Pat. No. 5,614,169, U.S. Pat. No. 5,679,459, U.S. Pat. No. 5,707,607, U.S. Pat. No. 5,711,933, U.S. Pat. No. 5,720,938, U.S. Pat. No. 6,001,335, U.S. Pat. No. 6,068,600, U.S. Pat. No. 6,132,699, U.S. Pat. No. 6,146,657, U.S. Pat. No. 6,165,442, U.S. Pat. No. 6,183,725, U.S. Pat. No. 6,193,952).

Moreover, the administration of therapeutic and diagnostic devices and medications or other active ingredients together with ultrasound contrast media into the human body has become known. U.S. Pat. No. 4,805,628 discloses a method, with which devices that can be implanted or inserted into the body, can be better visualized using ultrasound. In this case, these devices contain air-filled spaces.

A fluid that contains gases and medications is described in U.S. Pat. No. 5,315,998. The administration of an ultrasound contrast medium, which consists of gas-filled microbeads, together with medications as a therapeutic system of medication administration, by means of which a large number of medications shall be administered, is described in U.S. Pat. No. 5,770,222. U.S. Pat. No. 5,849,727 and U.S. Pat. No. 6,117,858 disclose a method, with which biological agents are administered to specific target structures. In this case, this substance used contains a solution of gas-filled microbubbles that are encapsulated by proteins, and a biological agent, which is either naproxen, piroxicam, warfarin or another defined substance. The gas used is a perfluorocarbon or sulfur hexafluoride.

Administering a photoactive substance, a gas, a precursor substance of a gas as well as a stabilizing material by means of ultrasound has become known from U.S. Pat. No. 6,123,923. U.S. Pat. No. 6,139,819 discloses a method, with which the internal organs of a patient with cardiac arrhythmia can be examined. Here, an ultrasound contrast medium is used that contains liposomes in an aqueous carrier solution, which encapsulate a fluorinated gas. At the same time, this ultrasound contrast medium contains a ligand, which binds to target cells or receptors, which are found in clots. The fluorinated gas used is either a perfluorocarbon or sulfur hexafluoride.

It should be emphasized that all the conventional techniques for using ultrasound contrast media are restricted to the forms of administration mentioned.

Introducing, e.g., cells or fillers into the human body for cosmetic or medical purposes has become known. The precise administration of suspensions of cells, viruses or fillers to a certain location in an organ has not been accomplished up to now with full satisfaction. The administrations take place frequently under endoscopic control (e.g., in case of administration in hollow organs), by means of a simple syringe in the case of administrations through the skin or within the framework of invasive surgeries. The distribution of the suspension in the tissue cannot be observed or documented in this case. According to the state of the art, cells are frequently administered together with biomaterials (e.g., collagens) or even with biocompatible materials. Insufficiencies in the precise administration also apply to these forms of administration [sic, typo in original—Tr.Ed.] of the suspensions of cells, viruses or fillers.

The administration of cells by means of an ultrasound probe is described in the unpublished patent application DE 100 58 370. However, the procedure of injecting cells into tissue under ultrasound control may, especially in small quantities, be perceived only insufficiently, since the ultrasound contrast of the tissue and of the cell suspension differ little. The size and especially the distribution of the cells (viruses, bacteria) in the tissue may therefore be followed only insufficiently. This is particularly disadvantageous if the cell growth is to be stimulated by the injection of cells in certain organs at a defined site, or if damaged tissue of a certain location in the organ in question is to be replaced by the injected cells.

The object of the present invention is to provide an improved material composition, with which the administration of biomaterials into tissue can be carried out with high accuracy and with improved effectiveness. Another object of the present invention is to indicate processes and devices for the production of the suspension composition. Another object of the present invention is to indicate forms of administration for suspension compositions and uses of the suspension compositions, and especially in the implantation of biomaterials and the treatment of tissue. Finally, the object of the present invention is to provide an improved process for the imaging of biological tissue.

These objects are accomplished by a suspension composition, processes and devices having the features according to the patent claims 1, 22, 27, 29, 34, 36 or 40.

Advantageous embodiments and uses of the present invention emerge from the dependent claims.

The basic idea of the present invention is to provide a suspension composition for administration in biological tissue, which contains at least one functional component in a suspension fluid in the form of at least one active ingredient, biological cells, cell constituents, microorganisms and/or biological or biocompatible fillers and at least one ultrasound contrast medium. By means of this combination a fundamentally novel use of ultrasound contrast media is achieved. Up to now contrast media were used only for the visualization of an organ (e.g., vein, internal organ). Contrary to this, for the first time, the suspension composition according to the present invention makes possible the ultrasound monitoring of the supply of at first exogenous material into a body part, especially tissue.

According to an advantageous use of the present invention, a suspension composition that contains at least one biological or biocompatible filler (e.g., collagens) and at least one ultrasound contrast medium is provided. The supply of fillers has particular advantages in medical treatments (e.g., of the urethra) or cosmetic processes (e.g., injecting under wrinkles).

According to another advantageous application of the present invention, a suspension composition is provided that contains at least one active ingredient and at least one ultrasound contrast medium. The at least one active ingredient is directed at biologically or medically effective changes in the tissue, in which the suspension composition is administered, or at another site in the body, at which the active ingredient is released. The supply of active ingredients in combination with ultrasound contrast media has particular advantages in relation to the targeted placing of the active ingredients in a tissue.

The active ingredient preferably contains a growth factor, such as, e.g., VEGF or EGF, which brings about a capillarization of biological tissue. The suspension composition according to the present invention makes possible an ultrasound-targeted modification of the tissue for the first time. The suspension composition acts like a therapeutic agent.

Depending on use, the active ingredient may be contained, e.g., in microbubbles that are contained in the suspension composition according to the present invention. The microbubbles consist, e.g., of a biocompatible material, such as, e.g., a protein, a lipid or a polymer, and may simultaneously act as ultrasound contrast medium. Thus, the present invention makes possible the administration of active ingredients, and in particular growth factors, enclosed in ultrasound contrast media. E.g., a quick vascularization of tissue sections, into which the suspension composition according to the present invention has been injected, is induced by the VEGF (vascular endothelial growth factor) or EGF (endothelial growth factor). This vascularization was not possible in an ultrasound-targeted manner using conventional techniques, in which contrast media were administered intravascularly, and in particular intravenously, since the active ingredients were distributed over the respective vascular system and did not remain at the injection site.

The vascularization brought about with a suspension composition according to the present invention yields particular advantages when injecting into hypoxic tissue sections, e.g., after heart infarction, or into tissue sections, into which cells are injected for therapeutic purposes. Vascularization is an important requirement for the restoration of the tissue or for the survival of cells in the tissue in the hypoxic area. A quick supply of blood to the modified area is guaranteed by the formation of capillaries in the tissue.

The present invention leads to an expanded use of ultrasound contrast media in comparison to the conventional intravascular administration. The ultrasound-targeted administration of the suspension compositions according to the present invention in the tissue makes possible a concentrated release of active ingredients, optionally in combination with other functional components, at the desired site in the body. Compared to administrations of biologically active substances and drugs in liposomes known up to now, the injection site and the kinetics of the release of the active ingredient can be documented exactly by means of the combination with ultrasound contrast media. By administering the active ingredients in microbubbles, which serve as ultrasound contrast media at the same time, it can be determined by ultrasound observation where an active ingredient is released. Only the intact microbubbles, in which the active ingredient is still contained, have a contrast-enhancing action. If the bubbles disintegrate, the included substances are released. The ultrasound contrast in the tissue area in question decreases. The bubbles may disintegrate spontaneously or induced by ultrasound.

The present invention pertains especially to the composition, production and use of a suspension of cells, viruses and/or bacteria that contains an ultrasound contrast medium. By means of this novel therapeutic agent, the requirements that live cells can be administered into the body in a manner targeted or monitored by ultrasound are met. By means of the ultrasound contrast medium and cells being injected at the same time, the injection can be observed and be documented using ultrasound techniques. This technique makes it possible to place even the smallest quantities of cells at an exact point in the body of a patient. Moreover, the position of the administered depot of cells can be checked and thus the therapeutic success can be documented and checked. With this novel administration technique, cells, which are in a suspension that contains one or more ultrasound contrast media as well as additional biomaterials (e.g., extracellular matrix) and/or may contain biocompatible materials, are administered exactly into the target organs together with ultrasound contrast media in a minimally invasive procedure (without highly invasive surgery and exposure of the target organs). The possibilities for using the so-called “tissue engineering” are thus decisively expanded.

According to another advantageous use of the present invention, the ultrasound contrast medium in the suspension composition according to the present invention is combined with biological cells that are provided as the functional component. The ultrasound contrast medium is contained, e.g., in the cells. This embodiment of the present invention again results in the particular advantage of an ultrasound-targeted observation of the injection result.

It was advantageously shown that the ultrasound contrast media administered with the suspension are broken down and/or are evacuated locally in the body. E.g., the ultrasound contrast medium combined with biological cells is transported into the body.

The co-administration of cells, additives and components, excipients and ultrasound contrast media represents a marked progress in the administration of cells for the treatment of a large number of diseases. Moreover, the production and provision of the therapeutic dosage form according to the present invention in many cases provides, first, the basis for the meaningful use of the technique of “tissue engineering” in diseases in hollow organs, body cavities, vessels, joints, internal organs and even in invasive surgeries.

Another subject of the present invention is a process for producing the suspension composition, which is especially characterized in that the at least one functional component and the at least one ultrasound contrast medium are mixed before or during the injection into the body. According to the present invention, the at least one functional component is mixed with a contrast medium solution or suspension while providing such concentration ratios that the suspension composition administered into the body has the functional component concentration that is correct for the action intended in each case and an osmolality adapted to the injection site. The at least one functional component is provided in the dissolved or suspended form for mixing or, as an alternative, in the solvent-free or low-solvent form (e.g., as centrifuged pellets). In the latter case, a centrifuged cell pellet is, e.g., added to the contrast medium solution or suspension. For loading biological cells with the ultrasound contrast medium, a culturing may be carried out in the presence of the contrast medium. E.g., macrophages take up the ultrasound contrast medium during the culturing. The macrophages are transferred into the suspension composition according to the present invention with the ultrasound contrast medium as the functional component.

Unlike conventional contrast media, which are administered in hyperosmolar solutions, e.g., into the blood stream, the osmolality of the contrast medium solution or suspension for the production of the suspension composition according to the present invention is set high. The mixing is carried out in such a way that the osmolality of the suspension composition preferably ranges from 340 mOsm/kg to 380 mOsm/kg, and especially preferably 350 mOsm/kg to 360 mOsm/kg. The mixing is carried out in a sterile container and/or in a mixing and/or administration means, which is additionally set up for the injection of the suspension composition into a body.

The setting of a high osmolality has the particular advantage that the functional component (and in particular cells) can be added into the suspension solution in a single step, without being damaged.

The ultrasound contrast medium is provided, e.g., dissolved or suspended in water. However, it is preferable to provide it in a physiological electrolyte or saline solution, which especially contains NaCl and KCl. The electrolyte solution contains, e.g., 140 mM of NaCl [sic] and 5 mM of KCl. As an alternative, a phosphate-buffered saline solution (PBS) may be provided. Especially preferred is the use of a saline solution that contains at least sodium and potassium ions, but preferably sodium, potassium, calcium and magnesium ions. Additives, such as, e.g., glucose, vitamins or pH-buffers may also be contained in the saline solution.

The composition mentioned here has the advantage that the functional component does not undergo any change due to osmosis and ion extraction during the formulation of the suspension composition according to the present invention. If the functional component consists, e.g., of biological cells, bacteria or viruses, an osmotic shock is avoided. The functional component survives the uptake into the suspension solution and the storage before the use on the tissue.

Another aspect of the present invention lies in the provision of a suspension kit (or: administration kit, suspension set, suspension packet or suspension container kit). The suspension kit according to the present invention is characterized by an arrangement of one or more containers, which contains or contain the suspension composition according to the present invention in the mixed state or in the state separated by components and is set up for insertion into a mixing and administration means. In the suspension kit, the components of the suspension composition are contained in preset ratios. The suspension kit makes possible a simplified and error-free supply of biomaterial suspensions into the tissue to be treated. According to a particular embodiment, the suspension kit may also contain only one container with at least one ultrasound contrast medium in a physiological solution or suspension. This suspension kit is used for the combination with low-media or media-free functional components (e.g., pellets).

Another subject of the present invention is a process for supplying the said suspension composition according to the present invention in endogenous tissue for therapeutic or cosmetic purposes. The injection is preferably carried out with a syringe needle or with a catheter, whereby the suspension composition according to the present invention is guided through the respective tool as a stream of fluid or in the encapsulated form.

According to an especially preferred embodiment of the present invention, the suspension composition is supplied using an ultrasound probe, as it is described in the unpublished German patent application DE 100 58 370, optionally in combination with an endoscope means.

Another subject of the present invention is a process for the modification of biological cells with an ultrasound contrast medium, in which this [medium] is taken up by the cells. The biological cells preferably contain cells that are effective in the immune system of a body. The ultrasound contrast medium is loaded by means of incubating the cells with the contrast medium in a medium, e.g., in a culturing vessel in a nutrient solution. The modified cells are used as the functional component of the above-described suspension composition. The uptake of synthetic particles in macrophages is described, e.g., by Dayton et al. in Biophysical Journal, Vol. 80, pp. 1547-1556, 2001.

In particular, a novel process for taking up ultrasound contrast media is made possible by means of the modification of cells according to the present invention. The ultrasound contrast media are optionally loaded with active ingredients. Advantageously, the loaded cells can be detected in the body with high-resolution ultrasound processes. E.g., the modified cells may migrate into the immune system of the body and be accumulated in the lymph nodes.

Another subject of the present invention is a process for imaging with an ultrasound imaging means, in which at least one ultrasound imaging of the tissue or of parts of this is carried out after or during an injection of the suspension composition according to the present invention in biological tissue. The process according to the present invention has the advantage that distribution of the suspension composition in the tissue can be determined, e.g., when a depot of the functional component forms. According to a preferred design of the process, e.g., geometric properties of the distribution and/or its temporal development are followed. The images determined according to the present invention are used as intermediate results, on the basis of which subsequent steps of medical diagnosis or treatment can be selected or carried out.

In an imaging process according to the present invention, any ultrasound imaging means can be used, as it is known per se from medical engineering. The imaging is preferably carried out after or during the injection of the suspension composition according to the present invention into the urethra at the urethral tissue or into the heart muscle.

Further advantages and details of the present invention shall become evident from the following description of the attached drawings, in which:

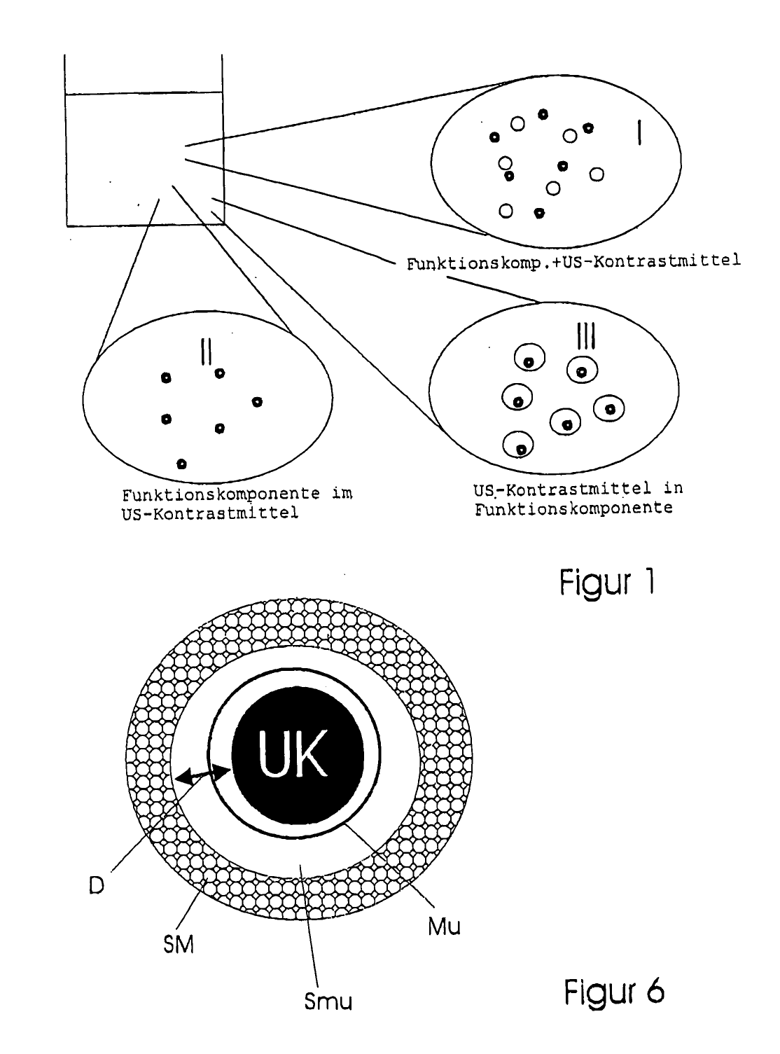

FIG. 1: shows a schematic illustration of various embodiments of suspension compositions according to the present invention,

FIGS. 2 through 5: show various embodiments of mixing and administration means for the production of suspension compositions according to the present invention, and

FIG. 6: shows a schematic sectional view of a urethra with an inserted ultrasound head.

The present invention is described below by way of example in relation to its use in the treatment of urinary incontinence. However, the implementation of the present invention is not limited to the exemplary embodiment described, but is also possible in other uses of administered biomaterials. Suspension compositions according to the present invention are generally produced from suspensions of at least one functional component, which is formed by the biomaterial to be administered, at least one ultrasound contrast medium and optionally at least one additive and active ingredient. Depending on the use, one or more of the components mentioned are selected from the following groups and are combined in a suspension composition.

Biologically Active Functional Component:

Biological cells, bacteria, viruses and/or biological or biocompatible fillers, and especially stem cells, precursor cells (e.g., myoblasts, fibroblasts, chondroblasts, neuroblasts, osteoblasts, cells of the hematogenic system), differentiated cells (e.g., chondrocytes, osteocytes, myocytes, fibrocytes, cells contained in blood, epithelial cells, hormonogenic cells, neurocytes, parenchymal cells from internal organs, endothelial cells, and cells of the immune system) or genetically altered cells or microorganisms.

Ultrasound Contrast Media:

The contrast media available per se are preferably used as ultrasound contrast media. These usually consist of suspended particles (e.g., bubbles) that are smaller than the red blood cells. By means of the particles, the ultrasound scattering is varied at the administration site, i.e., e.g., in the tissue. Although ultrasound contrast media were used for injection into tissue before the present invention and were particularly used to distinguish vessels from the surrounding solid tissue, it surprisingly emerged with the present invention that the ultrasound contrast media also lead to an evaluatable ultrasound contrast in the solid tissue. Generally, a distinction is made between two processes for producing contrast-giving microbubbles, namely the generation of free or sheathed gas bubbles. Depending on the stability of the sheathing substances, a distinction is made between readily soluble, poorly soluble or insoluble gas bubbles.

Especially used as ultrasound contrast media are: Free gas bubbles, precursor substances of gases, gas bubbles encapsulated by organic or inorganic substances, solutions, colloidal solutions, suspensions, dispersions, ionophoric agents, dissolved microparticles, dissolved polymer beads, dissolved organic and/or inorganic molecules, sugar-containing solutions, microparticles, ferro- or paramagnetic metals, microaggregates, porous particles of organic and/or inorganic material, lipid-containing microbeads and/or emulsions, whereby the gas bubbles preferably contain air, nitrogen, oxygen, carbon dioxide, fluorinated gas and/or another biocompatible gas.

Additives and Active Ingredients:

Salts, carbohydrates, e.g., dextrose, lipids, proteins, lipoproteins, one or more amino acids, fatty acids, simple sugar molecules, growth factors, hormones, iron, biologically active cell mediators, blood derivatives, enzymes, vitamins, peptides, bulking agents, biocompatible polymers (such as, e.g., collagen, hyaluronic acid, synthetic polymers or fibrin), matrix molecules of organic and/or inorganic material, a porous solid matrix, messenger substances, neurotransmitters and/or medications (especially antibiotics, tuberculostatics, fungicides, antiallergy agents, antiviral substances, anticoagulants, thrombolytics, medications against protozoa, medications against arteriosclerosis, antirheumatics, narcotics, opiates, cardiac glycosides, vasoactive substances, metabolic potentiators, substances against angiogenesis, substances for angiogenesis, chemotactic substances, nerve growth factors, neuromuscular blockers, sedatives, local anesthetics, anesthetics, radioactive substances, antibodies, genetic material, RNA, DNA, antisense-RNA, antisense-DNA, ribozymes, antigenic nucleic acids, cytostatics, photoactive substances, photosensitizers, contrast media for x-ray radiation, contrast media for MRI examinations, immune-response-stimulating and/or immunosuppressive substances).

Suspended Fluid:

Synthetically produced solution (e.g., physiological saline solution) and/or tissue fluid consisting of human and/or animal material.

Preferred uses of the suspension composition according to the present invention are an improved imaging of biological tissue, especially in the live body.

Other preferred uses of the suspension composition according to the present invention are in the treatment of urinary incontinence and/or fecal incontinence, ureterovesical reflux, gastroesophageal reflux[,] volume defects and/or wrinkles in plastic surgery (e.g., by subcutaneous administration of fibroblasts or other cells), and/or muscle diseases, diseases of the respiratory tract, the genitourinary system and the gastrointestinal tract, diseases of the nervous system, organ diseases, diseases of the hematogenic system, diseases of the hormone system (such as, e.g., diabetes mellitus), tumors, inflammations, infections, arthroses and joint injuries, diseases of the dermis, epidermis and subcutis, and/or traumas and injured organs, bones, joints, ligaments and muscles.

Various embodiments of suspension compositions according to the present invention are illustrated schematically in FIG. 1. According to a first variant (I), the functional component and the at least one ultrasound contrast medium are contained as separate components in a suspension solution. This variant has the advantage that the suspension composition according to the present invention can be prepared and administered in a particularly simple manner. According to modified variants (II, III), the functional component and the ultrasound contrast medium are combined with one another, whereby the functional component is contained in the ultrasound contrast medium (II) or the ultrasound contrast medium is contained in the functional component (III). As an alternative, an adherent joining of the two components (not shown) would also be possible. The variants II and III have the advantage of an expanded applicability of the suspension composition.

The supply of the suspension composition with a mixing and administration means according to the present invention is explained below according to various exemplary embodiments.

With the mixing and administration means according to FIG. 2, the cells, the additives and active ingredients, the carrier medium and the ultrasound contrast medium which is used are mixed before the administration in a syringe-like fluid conveyor with a suspension cylinder Z. The suspension cylinder Z has an inner chamber that forms two partial volumes. In the exemplary embodiment that is shown, the contrast medium KM is located in a first partial volume between the piston K and the syringe needle S, while the second partial volume, which is also designated as volume reserve VR, is formed on the rear side of the piston K. For production of the suspension composition according to the present invention, the suspension of the at least one functional component and optionally the additives and active ingredients is drawn by the fluid conveyor into the suspension cylinder while actuating the piston K. During the drawing up of the suspension, the contrast medium KM and the drawn-up suspension are mixed with one another. The suspension composition is then injected into a tissue by the movement of the piston K via the syringe needle S. The ultrasound contrast medium consists in this case, e.g., of stable gas bubbles, which are enclosed by a lipid-containing membrane.

The concrete design of the fluid conveyor illustrated in FIG. 2 is selected as a function of the use. The size of the suspension cylinder is especially dependent on the quantities of the suspension to be administered depending on the case. Furthermore, an adaptation to additional devices, such as, e.g., endoscopy devices and/or an ultrasound probe is optionally provided. If used, e.g., in combination with the medical instrument according to the unpublished patent application PCT/EP 01/13535, the syringe needle S is lengthened for formation of the injection tool corresponding to the dimensions of the instrument.

According to a preferred design, the setup consisting of the suspension cylinder Z, the piston K and the contrast medium KM taken up in the first partial volume with a projection for a syringe needle, which is closed at first, forms a ready-to-use kit for the production of the suspension composition.

One proceeds, e.g., as follows to provide a suspension composition containing biological cells and an ultrasound contrast medium. The cells are removed from an incubator. The cell culture medium is centrifuged in a manner known per se. A cell pellet is present, which is added to the suspension solution containing the contrast medium. Deviating from the above-illustrated procedure, the pellet is introduced into the contrast medium KM, e.g., in an arrangement according to FIG. 2 with the piston K removed. Advantageously, the content of the contrast medium is so low in the suspension composition that the osmolality is hardly changed. The suspension solution has, e.g., a composition containing 140 mM of NaCl, 4 mM of KCl and 1.5 mM of CaCl₂. The pH value ranges, e.g., from 7 to 7.4.

The volume prepared for administration is selected as a function of use and ranges, e.g., from 10 μL to 2 mL.

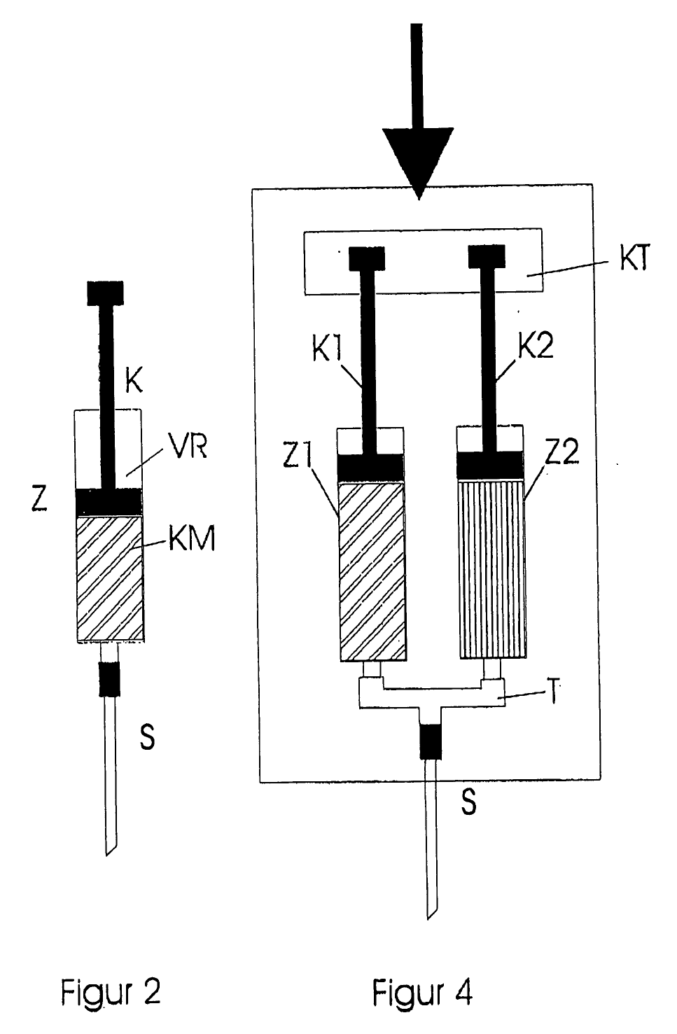

According to another exemplary embodiment shown in FIG. 3, the fluid conveyor has two chambers Ka1, Ka2 in the suspension cylinder Z. The chamber Ka1 is separated from the suspension cylinder Z by a container attached to the piston K, whose wall is formed by a membrane M (e.g., film) on the side pointing towards the chamber Ka2. A perforation or puncture device P, which is admitted into the piston K in a fluid-tight manner and which can be actuated from outside for breaking through the membrane M, runs in the piston K. The fluid conveyor with the suspension cylinder Z, piston K and the chamber Ka1 used again forms a ready-to-use kit for producing the suspension composition according to the present invention. The syringe needle S may optionally also be firmly attached to the kit according to the present invention. Depending on use, it is possible that the chamber Ka2 with the suspension of the at least one functional component is contained in the prepared kit, or the suspension is drawn up immediately before use by the syringe needle S. The functional component and the contrast medium are at first still separated by the membrane M. As a result, the mixing of the contrast medium and of the cell suspension does not occur immediately after the uptake, e.g., of suspended cells into the fluid conveyor. A particular advantage of this design lies in the fact that the cell suspension and the ultrasound contrast medium can be conveyed, without both components being mixed. The mixing takes place immediately before the administration. The thorough mixing of the ultrasound contrast medium and cells takes place, e.g., by passing through the membrane with the perforation device P.

Deviating from the view in FIG. 3, the perforation device P may be provided on other parts of the fluid conveyor, e.g., on the side of the chamber Ka2.

According to another exemplary embodiment (not shown), the suspended cells, the additives and active ingredients, the suspending or carrier agent and the ultrasound contrast medium used are only mixed together in a syringe immediately before administration. The ultrasound contrast medium K used consists, e.g., of galactose [sic]-picric acid particles and is located in its own second container. Immediately before the injection, a syringe containing the cells, additives and active ingredients and the carrier agent and the vessel containing the ultrasound contrast medium are combined and are carefully thoroughly mixed. The resulting mixture is then drawn up in the syringe, which does not contain any gas bubbles now, and is set onto the injection needle.

According to the exemplary embodiment shown in FIG. 4, the suspended cells, the additives and active ingredients, the carrier agent and the suspended ultrasound contrast medium are located in two separate suspension cylinders Z1, Z2 of a fluid conveyor. The suspension cylinders Z1, Z2 are each set up with a piston K1, K2, as syringe cylinders known per se. The said pistons K1, K2 are connected by a coupling piece KT and can be moved at the same time in the direction of the arrow, so that the ultrasound contrast medium from the suspension cylinder Z1 and the cells from the suspension cylinder Z2 are mixed in the T-shaped connecting piece and leave the common syringe needle at the same time as a composition according to the present invention. Various mixing ratios of the individual components can be predetermined by selecting the respective syringe volumes.

The fluid conveyor according to FIG. 4 is prepared by using the suspension cylinders Z1, Z2 that are provided as a kit. As an alternative, it is also possible to equip the T-shaped connecting piece T with a valve arrangement and to charge the suspension cylinders Z1, Z2 separately via the syringe needle S.

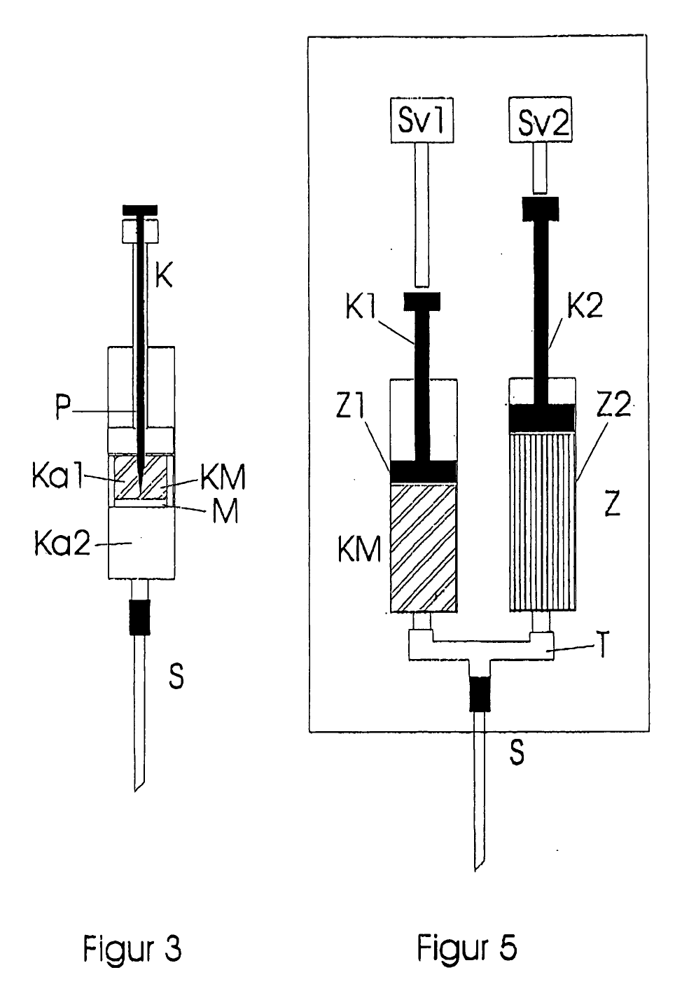

According to the exemplary embodiment shown in FIG. 5, the suspended cells, the additives and active ingredients, the carrier agent and the ultrasound contrast medium used are located in separate suspension cylinders Z1, Z2. Unlike the embodiment according to FIG. 4, the pistons K1, K2 of the suspension cylinders Z1, Z2 can be actuated separately with separate control devices SV1, SV2. The entire arrangement [sic, is?—Tr.Ed.] inserted into an electrically driven administration device AG. The individual components of the suspension composition according to the present invention are first mixed during the injection and injected into the body. The mixing ratio of the individual components is either preprogrammed or may be selected before the injection. The administration device contains a handle and one or more control units, which move the pistons.

A medical instrument, which can be especially advantageously used for injection of the suspension composition according to the present invention, is described in the unpublished patent application PCT/EP 01/13535, whose disclosure content is incorporated into the present patent application especially in relation to the setup of the instrument.

For imaging according to the present invention, for example, the said instrument, which is set up for injection and ultrasound measurement, is at first brought into position in the body part to be treated (e.g., hollow organ, tissue). The tissue to be treated is visually observed with an endoscopy device. An injection tool (e.g., syringe, hollow needle, catheter) is positioned with a control device or under ultrasound control. The said suspension composition is then administered via the tool into the tissue area in question. During and/or after the injection, the progress of the administration is monitored by imaging with an ultrasound probe.

The carrying out of the imaging process according to the present invention with the injection of the suspension composition in the human urethra is schematically illustrated in FIG. 6. An ultrasound head UK is arranged in the urethra, which is essentially arranged in layers consisting of the mucosa Mu, the submucosa Smu and the sphincter muscle SM. Myoblasts are injected with the suspension according to the present invention into the sphincter SM. The ultrasound head is a component of an ultrasound imaging means, with which the distribution of the suspension in the sphincter muscle is detected. The contractility of the urethra is improved by the supply of myoblasts. According to the present invention, a provision may be made to carry out contractility measurements immediately with the instrument, and especially with the ultrasound probe. For this, the distance D between the ultrasound head UK and the sphincter muscle SM is measured. Depending on the distance values that can be achieved, it is determined whether enough myoblasts were supplied or whether another injection must be carried out.

The features disclosed in the above specification, the claims and the drawings may be of importance both separately and combined for accomplishing the present invention in its various embodiments.

1. Suspension composition, which contains at least one functional component that is effective in biological tissue and forms a dosage form for administration into the tissue, whereby the said functional component is suspended or dissolved in a suspended fluid,
characterized in that
the functional component contains at least one active ingredient, biological cells, bacteria, viruses and/or biological or biocompatible fillers, and an ultrasound contrast medium is contained in the suspended fluid.

2. Suspension composition in accordance with claim 1, in which the biological cells include biologically active, live and/or dead cells.

3. Suspension composition in accordance with claim 2, in which the live cells include stem cells, precursor cells (e.g., myoblasts, fibroblasts, chondroblasts, neuroblasts, osteoblasts, cells of the hematogenic system), differentiated cells (e.g., chondrocytes, osteocytes, myocytes, fibrocytes, cells contained in blood, epithelial cells, hormonogenic cells, neurocytes, parenchymal cells from internal organs, endothelial cells, cells of the immune system), macrophages or genetically altered cells or microorganisms.

4. Suspension composition in accordance with one of the above claims, in which the suspended fluid is formed synthetically or from human or animal material, and especially by a physiological electrolyte solution.

5. Suspension composition in accordance with claim 4, in which the suspended fluid contains a physiological saline solution, which contains at least one salt of an element of the first main group and at least one salt of an element of the second main group.

6. Suspension composition in accordance with claim 5, in which the suspended fluid contains sodium, potassium, calcium and magnesium ions.

7. Suspension composition in accordance with one of the above claims that has an osmolality in the range of 340 mOsm/kg to 380 mOsm/kg.

8. Suspension composition in accordance with one of the above claims, in which the suspended fluid is additionally modified by means of additives and active ingredients.

9. Suspension composition in accordance with claim 8, in which the active ingredient or the additives and ingredients include salts, carbohydrates, lipids, proteins, lipoproteins, amino acids, fatty acids, simple sugar molecules, growth factors, hormones, biologically active cell mediators, blood derivatives, enzymes, vitamins, peptides, bulking agents, biocompatible polymers (such as, e.g., collagen, hyaluronic acid, synthetic polymers or fibrin), matrix molecules from organic and/or inorganic material, a porous solid matrix, messenger substances, neurotransmitters and/or medications (especially antibiotics, tuberculostatics, fungicides, antiallergy agents, antiviral substances, anticoagulants, thrombolytics, medications against protozoa, medications against arteriosclerosis, antirheumatics, narcotics, opiates, cardiac glycosides, vasoactive substances, metabolic potentiators, substances against angiogenesis, substances for angiogenesis, chemotactic substances, nerve growth factors, neuromuscular blockers, sedatives, local anesthetics, anesthetics, radioactive substances, antibodies, genetic material, RNA, DNA, antisense-RNA, antisense-DNA, ribozymes, antigenic nucleic acids, cytostatics, photoactive substances, photosensitizers, contrast media for x-ray radiation, contrast media for MRI examinations, immune-response-stimulating and/or immunosuppressive substances).

10. Suspension composition in accordance with one of the above claims, in which the active ingredient contains a growth factor, which brings about a capillarization of biological tissue.

11. Suspension composition in accordance with claim 10, in which the growth factor contains VEGF or EGF.

12. Suspension composition in accordance with one of the above claims, in which the functional component is contained in microbubbles, which are suspended in the suspended fluid.

13. Suspension composition in accordance with claim 12, in which the microbubbles consist of a material that can be destroyed by ultrasound irradiation.

14. Suspension composition in accordance with one of the above claims, in which the functional component and the ultrasound contrast medium form a bond.

15. Suspension composition in accordance with one of the above claims, in which the said ultrasound contrast medium is formed by free gas bubbles, precursor substances of gases, gas bubbles encapsulated by organic or inorganic substances, solutions, colloidal solutions, suspensions, dispersions, ionophoric agents, dissolved microparticles, dissolved polymer beads, dissolved organic and/or inorganic molecules, sugar-containing solutions, microparticles, ferro- or paramagnetic metals, microaggregates, porous particles of organic and/or inorganic material, lipid-containing microbeads and/or emulsions, which reflect, amplify or absorb ultrasound waves.

16. Suspension composition in accordance with claim 15, in which the gas bubbles of the said ultrasound contrast medium contain air, nitrogen, oxygen, carbon dioxide, fluorinated gas, another biocompatible gas and/or the at least one active ingredient.

17. Suspension composition in accordance with one of the above claims, in which the said ultrasound contrast medium is contained in biological cells, which are suspended as the functional component in the suspended fluid.

18. Suspension composition in accordance with claim 17, in which the biological cells contain macrophages.

19. Suspension composition in accordance with one of the above claims, in which the said ultrasound contrast medium takes up less than half of the volume content of the suspended fluid containing the functional component.

20. Suspension composition in accordance with one of the above claims, in which the functional component contains a substance that is suitable for the treatment of
urinary incontinence and/or fecal incontinence,
ureterovesical reflux, gastroesophageal reflux[,] volume defects and/or wrinkles in plastic surgery (e.g., by means of subcutaneous administration of fibroblasts or other cells), and/or
muscle diseases, diseases of the respiratory tract, genitourinary system and gastrointestinal tract, diseases of the nervous system, organ diseases, diseases of the hematogenic system, diseases of the hormone system (such as, e.g., diabetes mellitus), tumors, inflammations, infections, arthroses and joint injuries, diseases of the dermis, epidermis and subcutis, and/or
traumas and injured organs, bones, joints, ligaments and muscles.

21. Suspension composition in accordance with one of the above claims, which is contained in a biocompatible and/or resorbable capsule.

22. Process for the production of a suspension composition in accordance with one of the above claims, in which all of the components are mixed either before the administration of the functional component to be administered or in which individual or all components are mixed only immediately before or during the injection into the body.

23. Process in accordance with claim 22, in which a predetermined volume of a suspension of the at least one functional component and a predetermined volume of the ultrasound contrast medium are mixed and introduced into a sterile container.

24. Process in accordance with claim 22 or 23, in which the mixing is carried out using a mixing and administration means with at least one suspension cylinder, whereby the ultrasound contrast medium is contained in a first partial volume of the at least one suspension cylinder and the suspension cylinder contains a second partial volume as a volume reserve, in which the suspension containing the functional component is taken up.

25. Process in accordance with claim 24, in which a mixing and administration means having a plurality of suspension cylinders is used, each of which forms a partial volume and correspondingly contain a suspension of the ultrasound contrast medium and the suspension of the functional component, whereby the suspension cylinders are emptied into a syringe needle, a catheter or a compressed air system for mixing at the same time.

26. Process in accordance with claim 25, in which the ratio of the emptying rates of the said suspension cylinders is preset as fixed and/or is controlled with a control means of the mixing and administration means.

27. Process for the production of a suspension composition in accordance with one of the above claims 1 through 21 by means of a syringe-type mixing and administration means, which consists of a syringe-like volume conveyor, which has a volume reserve, so that, after drawing up a suspension of the functional component, a volume content of the ultrasound contrast medium can still be drawn up, or after drawing up the ultrasound contrast medium, a volume content of the suspension of the functional component can be drawn up, such that the suspension is ready to use after the thorough mixing of the components.

28. Process in accordance with claim 27, in which the individual components are contained in one, two or more syringe systems arranged in parallel or in series, which, in the administration, at the same time administer the contained components with a syringe needle, a catheter, a compressed air system or a surgical device into the body, whereby the ratio of the components that are to be administered is either preset as fixed before the administration or is selected by means of a control device of the injection system.

29. Process for the injection of a suspension composition in accordance with one of the claims 1 through 21 in biological tissue, whereby the suspension composition is administered by means of a nozzle into extravascular tissue outside of the vascular system of a body using an injection needle, a catheter or by means of compressed air acceleration in a manner monitored by ultrasound.

30. Process in accordance with claim 29, in which the suspension composition is administered into tissue of walls of hollow organs, such as, e.g., in the gastrointestinal tract, genitourinary tract, nose and paranasal sinuses, respiratory tract, joints, body cavities, such as, e.g., abdominal cavity, chest cavity, brain cavity, pelvic cavity, organs, such as, e.g., the brain, pancreas or liver, a puncture track, vessels, muscle tissue, connective tissue, bones, cartilage or under the skin or intraoperatively in an operating room.

31. Process in accordance with claim 29 or 30, in which the suspension composition is packed into a biocompatible and/or resorbable capsule and is administered with a corresponding injection needle or a catheter.

32. Process in accordance with one of the claims 29 through 31, in which vascularization of the tissue is induced at the site of administration of the suspension composition.

33. Process in accordance with one of the claims 29 through 32, in which the said suspension composition contains active ingredients, which are arranged in microbubbles, and in which the active ingredients are released in the tissue in a manner induced by ultrasound.

34. Administration kit having one or more suspension cylinders, which contain a suspension composition in accordance with one of the claims 1 through 21 or its components separately.

35. Administration kit in accordance with claim 34, in which the at least one suspension cylinder is equipped with a piston and a projection for a syringe needle or a catheter.

36. Process for the modification of biological cells, in which an ultrasound contrast medium is taken up by the biological cells.

37. Process in accordance with claim 36, in which the uptake of the ultrasound contrast medium takes place by incubation of the cells and addition of the ultrasound contrast medium.

38. Process in accordance with claim 36 or 37, in which the said biological cells contain macrophages.

39. Use of a physiological saline solution that contains ions of elements of the first and second main groups and has an osmolality ranging from 340 mOsm/kg to 380 mOsm/kg for the production of a suspension composition in accordance with one of the claims 1 through 21.

40. Process for imaging with an ultrasound imaging means, with the steps:
injection of a suspension composition in accordance with one of the claims 1 through 21 into biological tissue, and
imaging of the tissue or of parts of the tissue with the ultrasound imaging means.

41. Process in accordance with claim 40, in which geometric properties of the distribution of the suspension composition in the tissue are determined.

42. Process in accordance with claim 40 or 41, in which repeated images of the tissue are taken after the injection and the temporal development of the distribution of the suspension composition in the tissue is determined.

43. Process in accordance with one of the claims 40 through 42, in which the tissue of the urethra or of the heart muscle is injected and imaged.
